(19) United States
(12) Patent Application Publication
(10) Pub. No.: US 2016/0140864 A1
(43) Pub. Date: May 19, 2016

(54) SYSTEM AND METHOD FOR MANAGING BINGE EATING DISORDERS

(71) Applicants: Evan M. Forman, Wynnewood, PA (US); Gaurav Naik, Elmwood Park, NJ (US); Jeffrey Segall, Philadelphia, PA (US); Meghan Butryn, Philadelphia, PA (US); Adrienne Juarascio, Philadelphia, PA (US); Stephanie Manasse, Philadelphia, PA (US); Stephanie Goldstein, Philadelphia, PA (US)

(72) Inventors: Evan M. Forman, Wynnewood, PA (US); Gaurav Naik, Elmwood Park, NJ (US); Jeffrey Segall, Philadelphia, PA (US); Meghan Butryn, Philadelphia, PA (US); Adrienne Juarascio, Philadelphia, PA (US); Stephanie Manasse, Philadelphia, PA (US); Stephanie Goldstein, Philadelphia, PA (US)

(73) Assignee: Drexel University, Philadelphia, PA (US)

(21) Appl. No.: 15/003,078

(22) Filed: Jan. 21, 2016

Related U.S. Application Data
(63) Continuation of application No. PCT/US2014/049772, filed on Aug. 5, 2014.
(60) Provisional application No. 61/862,362, filed on Aug. 5, 2013.

(51) Int. Cl. G09B 19/00; A61B 5/00; A61B 5/11

(52) U.S. Cl. CPC G09B 19/00; A61B 5/1118; A61B 5/4833; A61B 5/486; A61B 5/4866; A61B 5/4869; A61B 5/1112; A61B 5/746; A61B 5/742; A61B 5/6831

(57) ABSTRACT

Systems, methods, devices, and applications for managing a binge eating disorder (BED). A smartphone and server application may be provided for monitoring and treating BED. Ecological Momentary Assessment (EMA) may be used for automated and/or manual acquisition of data related to triggers associated with BED. Risk factors may be captured in real-time to provide data to both patients and clinicians. For manual data acquisition, the user may be prompted to input emotional states, urges to binge, eating behavior, and binge episodes. For automated acquisition, time, place (e.g. via GPS), medicine adherence, and physical activity may be recorded. The user may be provided with alerts and/or interventions when it is determined that they are at risk of binging.

### CROSS-REFERENCE TO RELATED APPLICATIONS

This application is a continuation under 35 U.S.C. §120 of International Application No. PCT/US2014/049772, filed Aug. 5, 2014, which claims priority to U.S. Provisional Patent Application Ser. No. 61/862,362, filed Aug. 5, 2013. The entire content of each of these applications is hereby incorporated by reference herein.

### FIELD OF INVENTION

The invention described herein is related to medical systems.

### BACKGROUND

Binge eating may be characterized as eating an unusually large amount of food within a short amount of time, accompanied by a subjective sense of loss of control over eating. Diagnostic criteria for binge eating disorder (BED) have typically associated BED with emotional distress, as occurring regularly, and as persistent. BED is the most common eating disorder in the United States, affecting 3.5% of females and 2% of males. Individuals diagnosed with BED exhibit high rates of psychiatric comorbidity, impairments in work and social functioning, reduced quality of life, and medical complications related to obesity.

A mix of factors may contribute to the onset and maintenance of BED, including stress, emotional dysregulation, interpersonal problems, low self-esteem, obesity and/or shape/weight distress. FIG. 1 is a diagram showing psychological and psychosocial factors that may contribute to the onset and maintenance of BED.

These factors may create an acute vulnerability to binge episodes. Such binges may occur as a result of specific internal (cognitive, affective or physiological) or external (social, environmental or object-based) triggers. Such triggers or cues may lead to maladaptive thoughts, feelings, and urges that lead to episodes of binge eating. Table 1 describes various internal and external triggers of maladaptive thought processes that may lead to binge eating.

TABLE 1
Description of Internal and External Triggers.

| Internal triggers | External Triggers |
|---|---|
| Cognitive: a memory, wish (e.g. | Social or interpersonal: social |
| wishing to be thinner), or mental | isolation, being with someone who |
| picture/image. | engages in problem behavior |
| Affective: a mood state (e.g., | Environmental: Location (e.g. home, |
| anxiety or depression) | work, living room), extreme sensory |
| | simulation (e.g. disorganization) |
| Physiological: Hunger; activity | Specific object: hearing, seeing, |
| level (e.g. physical exhaustion); | smelling an object or thing (e.g., |
| proprioceptive changes (e.g., | smell of food, seeing an advertisement) |
| feeling full) | |

Much like other eating disorders, treatment of BED may be challenging due to feelings of guilt, shame, or denial about the disorder. Eating disorders may be treated with a variety of techniques, often using some combination of the following: group/family therapy; medication; nutritional counseling; and psychotherapy. The eventual goal of treating BED is to empower the affected person to control their eating behavior.

### SUMMARY

Systems, methods, devices, and a smartphone application for monitoring and treating Binge Eating Disorder (BED) which may include Ecological Momentary Assessment (EMA) for automated and manual acquisition of data related to triggers associated with BED. Triggers and risk factors may be captured “in the moment” to provide meaningful data to both patients and clinicians. The data gathered may fall into at least two (2) categories including manual acquisition and automated data acquisition. For manual data acquisition, the user may be prompted to input emotional states, urges to binge, eating behavior, and binge episodes. For automated acquisition, record time, place (e.g. via GPS), medicine adherence, and physical activity may be recorded.

Using the data gathered using EMA, Ecological Momentary Interventions (EMI) may be implemented by applying computer-based machine learning algorithms to an understanding of trigger-binge associations and trigger-binge associations learned from the user of the application, using machine learning for example. Thus the user may be provided with EMIs when it is determined that patient is at risk. The data gathered from each patient may be transmitted to a central server that applies other computer-based algorithms across all users to improve the system's capability to provide EMIs. These may include supervised learning methods such as Support Vector Machines (SVM) and k-nearest neighbor.

User-directed coping strategies, self-directed help modules, and social connectivity may also be provided to the user. Automated connectivity to external hardware devices such as fitness tracking wristbands, smart pill bottle caps, and Wi-Fi connected body weight scales may be used for automatic collection of user data used in EMA.

### BRIEF DESCRIPTION OF THE DRAWINGS

A more detailed understanding may be had from the following description, given by way of example in conjunction with the accompanying drawings wherein:

FIG. 1 is a diagram illustrating psychological and psychosocial factors that may contribute to the onset and maintenance of BED;

FIG. 2 illustrates an overview of an example system for treating BED;

FIG. 3 illustrates an example embodiment of the system of FIG. 2;

FIG. 4 illustrates examples of peripheral devices capable of communication with the system of FIG. 3;

FIGS. 5-15B illustrate screen shots of an example user application operating on a mobile user device of the system of FIG. 3; and

FIG. 16 illustrates an example architecture of the mobile user device of FIGS. 3-15B.

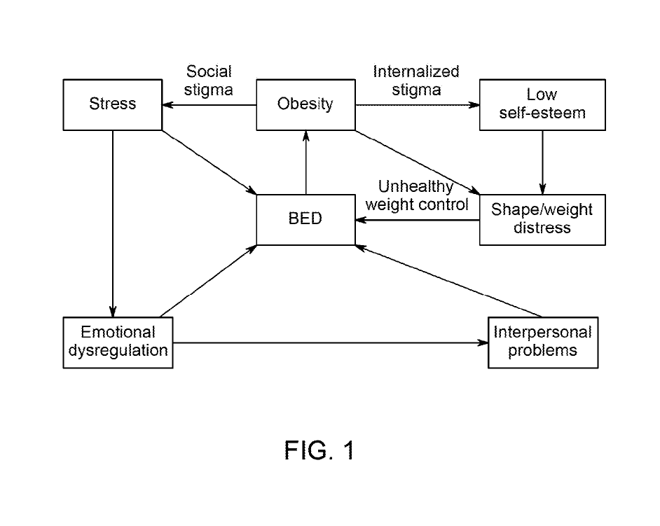

### DETAILED DESCRIPTION

Identifying and responding to triggers has been identified empirically as a critical component of efficacious interventions for BED. Accordingly, systems, methods, and devices for treating BED are described herein which may analyze relationships between binge triggers and binge episodes, and may deliver customized, automated and/or self-selected interventions to a user. Such interventions may include one or more interventions found in CBT for BED, (See Table 2).

Table 2 lists various interventions for BED which may be found in CBT.

| Stages of CBT | |
|---|---|
| for BED | Goals of treatment |
| 1 | Behavior and cognitive strategies to target binge eating |
| | Stabilization eating patterns through use of behavioral |
| | strategies |
| | Establish pattern of self-monitoring |
| | Identification of binge eating cues/triggers and |
| | consequences |
| | Cognitive restructuring |
| 2 | Addressing associated problems that accompany binge |
| | eating behavior |
| | Strategies for self-control and mood enhancement |
| | Improving body image, self-esteem |
| | Stress management and problem solving |
| 3 | Maintaining improvement and preventing relapse |
| | Planning to prevent relapse |
| | Practicing exposure to high risk foods and situations |
| | Review of progress and planning a healthy lifestyle |

Such systems, devices, and methods may be used as a guided self-help treatment in conjunction with pharmacotherapy and/or assistance from a health care professional, and may include a user application (“app”) which may be smart-phone based, or may include any other suitable computing or communications devices and methods. The app and/or other system components may provide auto-generated interventions to discourage binge eating, user-requested coping strategies, self-directed CBT modules for BED, social connectivity, healthy lifestyle interventions, data visualization, and/or a clinician portal as further described herein.

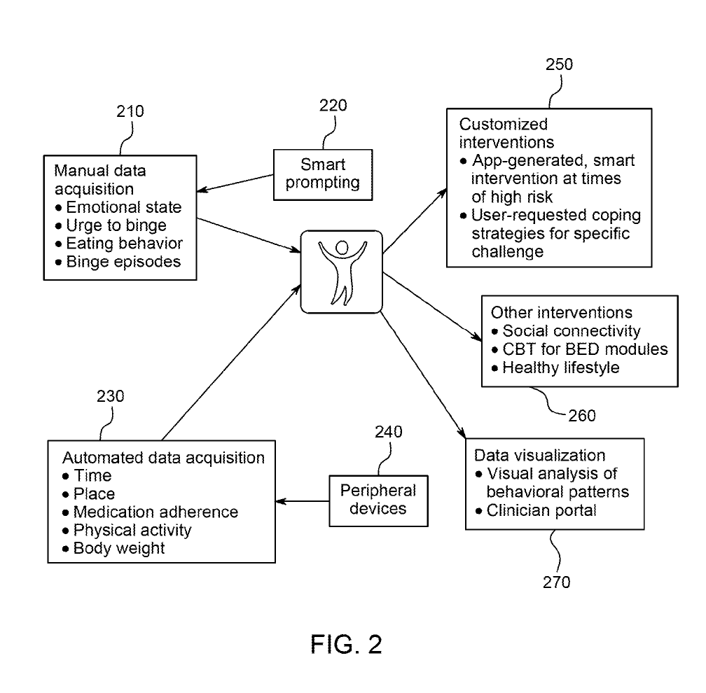

FIG. 2 is a chart illustrating a high level view of an example system 200 for managing binge eating disorders. The system may facilitate dissemination of an evidence-based treatment, for example, to patients who do not have access to BED specialists. As shown in FIG. 2, system 200 may include a manual data acquisition module 210 (including a smart prompting module 220), an automated data acquisition module 230, one or more peripheral devices 240, a customized interventions module 250, an other interventions module 260, and a data visualization module 270. Each of these modules may be configured to communicate with the user interface of the system 200.

The manual data acquisition module 210 may include any suitable interface, such as a mobile device touch screen, configured to receive data inputs from the user, such as emotional state, urge to binge, eating behavior, episodes, or other data, possibly in response to “smart” context-aware or other prompts 220 as discussed herein. Automated data acquisition module 230 may include any suitable interface, such as a BLUETOOTH® or other communications interface, for receiving data from peripheral or other devices 240 relating to time, place, medication adherence, physical activity, body weight, or other data as discussed herein. Customized interventions module 250 may include a processing module configured to generate alerts, messages, or other interventions based on an analysis of the acquired data and/or other information as discussed herein. Other interventions module 260 may include a processing module configured to allow a user to engage in self-directed interventions such as engaging in social connectivity, CBT, or receiving healthy lifestyle guidance as discussed herein. Data visualization module 270 may include a processing module configured to present charts, graphs, statistics, analyses, or other visualizations of user behavior to a user or clinician on a display as discussed herein, which may assist the user or clinicians in monitoring treatment of BED.

These various components may be implemented using Ecological Momentary Assessment; Ecological Momentary Intervention; guided, modular and/or other self-help; social support (e.g. via electronic social networks); data visualization, and/or a clinician portal as further described herein.

Ecological Momentary Assessment (EMA) may include assessment or repeated assessment of current (or recent) behaviors and psychological processes in their real-world context. EMA may produce accurate and rich data sets, and capture temporal antecedents and consequences of behavior. Implementations may be configured for easy and/or one-touch manual tagging of certain triggers (e.g., emotional, cognitive, interpersonal, situational) as they occur or thereafter using manual data acquisition module 210, as well as automated collection of other triggers (e.g. time and place, via time stamping and geolocation) using automated data acquisition module 230. Implementations of manual data acquisition module 210 may incorporate an EMA app such as DREXELEMA™ in order to facilitate trigger tagging, such as for reporting of urges and behaviors.

Medication adherence, body weight, and exercise behavior may be self-reported using manual data acquisition module 210 or may be obtained through interconnectivity with peripherals such as wireless pillboxes, scales, wristband or other personal activity monitors, or smartphone sensors using automated data acquisition module 230. Examples of such peripherals include the FitBit Flex™ activity band, the WITHINGS™ wireless scale, VITALITY™ GLOWCAPS™ and ADHERETECH™ bottles. The system may receive data transmissions from such peripherals and may communicate with such peripherals using an open platform or other application programming interface (API).

Smart analytics and/or machine learning modules (not shown) may be used to analyze the collected data over time to predict binge eating episodes and/or to provide data visualization for patients and clinicians using data visualization module 270.

The system 200 may analyze the input data over time using smart analytics or machine learning modules (not shown) to build increasingly accurate associations between detected triggers and binge episodes, thus allowing for preventative interventions or other alerts to be automatically delivered to a user via customized interventions module 250. Such interventions and alerts may be presented in response to the presence of triggers detected by analyzing data from manual data acquisition module 210 or automated data acquisition module 230 which the system has determined are predictive of binges for that particular user. Thus, the system may auto-generate interventions using customized interventions module 250 (e.g. signaled through audio and visual means on a smartphone app or text message) when the patient enters a risky location (e.g., supermarket), engages in risky behavior (e.g., misses a dose of medication), possibly during a high-risk time period (e.g., midnight), and/or after a reported affect or emotion (e.g., shame) or interpersonal conflict as established by the analysis.

During detected or predicted high risk situations, an Ecological Momentary Intervention (EMI) may be provided to the user using customized interventions module 250 and/or other interventions module 260. The EMI may include customized interventions for BED which may be based on the principles of Cognitive Behavioral Therapy (CBT). Such tailored interventions, which may be delivered in the moment and in a real-world context, may engender patient engagement and may be more efficacious than interventions transmitted in an office setting.

The system 200 may match such interventions to risk categories (e.g. providing a cue to eat a snack when the patient has gone more than three hours without eating, rational responses to permissive cognitions, and coping strategies for high-risk times of day), and may adapt these interventions from an empirically-supported BED self-help treatment protocol, (e.g. Fairburn's Overcoming Binge Eating). The matching intervention (e.g., addressing event-related changes in eating such as binging in response to stress) may be presented in a step-by-step fashion by modules 250 and/or 260 (e.g., Screen 1: awareness of emotional and urge fluctuations; Screen 2: identifying and restructuring cognitions promoting emotion and urge to binge; Screen 3 coping strategies to substitute for binging). The user may be presented with a problem menu via manual data module 210 to allow the user to name a currently occurring problem (e.g., thoughts excusing a binge) and receive a matching strategy via modules 250 or 260. The system 200 may also request users to make predictions about their expectations of binges (e.g., extent to which a binge may improve affect) and to report actual post binge states using manual data acquisition module 210, so as to facilitate comparisons between expectations and realities (which are known to be discordant in BED).

Guided, modular, and/or other self-help may be provided to the user via modules 250 or 260. For example, material based on Fairburn's Guided Self-Help for BED protocol (or any other self-help protocol) may be provided on demand, such as by module (e.g., normalization of eating, self-monitoring, coping strategies for triggers/urges). The content may be interactive, and patient-entered information may be collected using manual data acquisition module 210 and retained in the system.

System 200 may include a social support module (not shown), and one or more types of social support may be provided via a social networking feature. The system 200 may prompt the user to interact with the social network in response to detected BED triggers using other interventions module 260. Patients may be permitted join a support community, which may for example include other sufferers of BED who are also using the system. System 200 may provide modules for permitting the support community to interact through instant messaging, app functionality, link sharing, or other social media interactions. System 200 may also permit users to self-designate usernames for the social community in order to provide patients with a desired level of anonymity, and to configure settings to permit certain elements of the patient's information (e.g., high-risk triggers) to be broadcast to the social support network (which the patient may specifically select), prompting others to offer encouragement at opportune moments. System 200 may provide modules which allow a user to request assistance from other users, either generally, or one-to-one. System 200 may also provide modules for the user to provide help to others via the social support features, which may be beneficial for the giver as well as the receiver.

System 200 may also include a clinician portal to provide clinicians with access to their patients' data, including: medication adherence, emotional states through time, binge episodes, trigger-binge associations, interventions attempted and responses to interventions. The portal may allow the clinician to assign specific parts of the intervention modules for transmission to the patient via customized or other interventions modules 250 and 260, to view the patients' adherence and responses to the interactive segments of the modules of system 200, and/or to send targeted supportive and skill-based messages.

System 200 may also include modules for providing incentives such as points, level attainment, and leaderboards to users, using customized or other interventions modules 250 and 260 for example, in order to encourage helping others in the network or other desired behaviors. Such incentives may be awarded on the basis of help tagged as useful by another user for example.

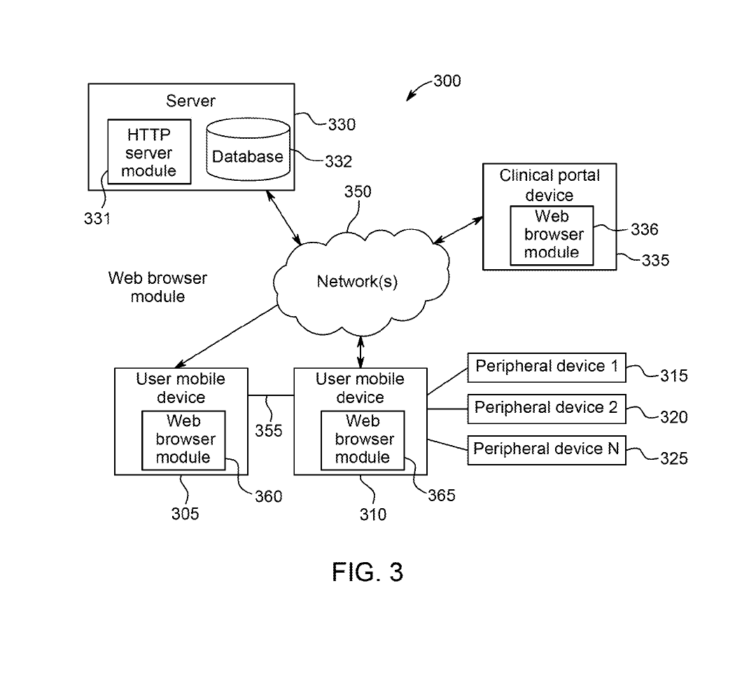

FIG. 3 illustrates an example system 300 for managing binge eating disorders which may be an implementation of system 200 described with respect to FIG. 2. System 300 includes mobile devices 305 and 310, peripheral devices 315, 320, and 325, server 330 and clinical portal device 335.

Network 350 may comprise one or more networks, and may include Wi-Fi based networks, cellular networks, BLUETOOTH®-based networks, or any other communications networks. The various devices connected to network 350 may communicate over network 350 using any communication protocols. Users may be able to communicate to other users directly (e.g. via peer-to-peer networks) or they may communicate via a standard Internet-based or other network.

Mobile devices 305 and 310 may include web browser modules 340 and 345 respectively and may communicate with each other via network 350 and/or via a peer-to-peer link 355. Mobile devices 305 and 310 may include a processor (not shown) and a non-transitory computer readable medium (not shown), and may store instructions on the computer readable medium which when executed by the processor cause the mobile device to carry out functionality of system 300 as described herein. Link 355 may include any suitable channel for direct communications between mobile device 305 and 310, such as a BLUETOOTH® connection. Mobile devices 305 and 310 may also communicate with server 330 and clinical portal device 335 via network 350. It is noted that other topologies may be used as appropriate.

Users of mobile devices 305 and 310 may interact with a program for managing binge eating disorders (not shown). The program may execute locally on mobile devices 305 and/or 310 as an application program, (“app”) or may be executed on a server 330. Server executed functionality may be accessed via a client program executing on mobile devices 305 and 310 such as an app (not shown) or web browser modules 360 and/or 365. It is noted that the program may be implemented using a hybrid local and server approach. For example, the program may execute some functions locally and others remotely on a server, or may store some data locally and other data remotely on the server. Web browser modules 340 and 345 may include any suitable client program for accessing a server such as server 330 over network 350.

Server 330 may be any suitable computing device for storing and analyzing data and/or hosting and serving web pages or other information for access over network 350. Server 330 may include a web server module 333 and a database 332. Server 330 may store data received from mobile devices 305 and 310 and peripheral devices 315, 320, and 325 in database 332 or another storage (not shown), may analyze the data, and/or may provide analysis or intervention alerts to mobile devices 305 and 310. Some or all of this functionality may instead be performed in mobile devices 305 and 310 in various implementations. Database 332 and/or other storage (not shown) may include a non-transitory computer readable medium, and may store instructions which when executed by a processor of server 330 cause server 330 to carry out functionality of system 300 as described herein.

Server 330 may host remote data collection and analysis functionality as described herein for access by mobile devices 305 and 310 over network 350. Server 330 may also host a clinical portal website as further described herein which may be accessed by a clinical portal device 335. The clinical portal may be configured to permit a clinician to access user data and analyses of user data, to assign interventions to users, and/or to permit messaging to users as described further herein.

Clinical portal device 335 may be any suitable computing or communications device for accessing the clinical portal website hosted by server 330, and may include a web browser module 336.

Peripheral devices 315, 320, 325 may include sensors for monitoring medication adherence, body weight, exercise behavior, or other information relating to BED or BED triggers, and may capture and report this data to mobile device 310 either automatically or on demand.

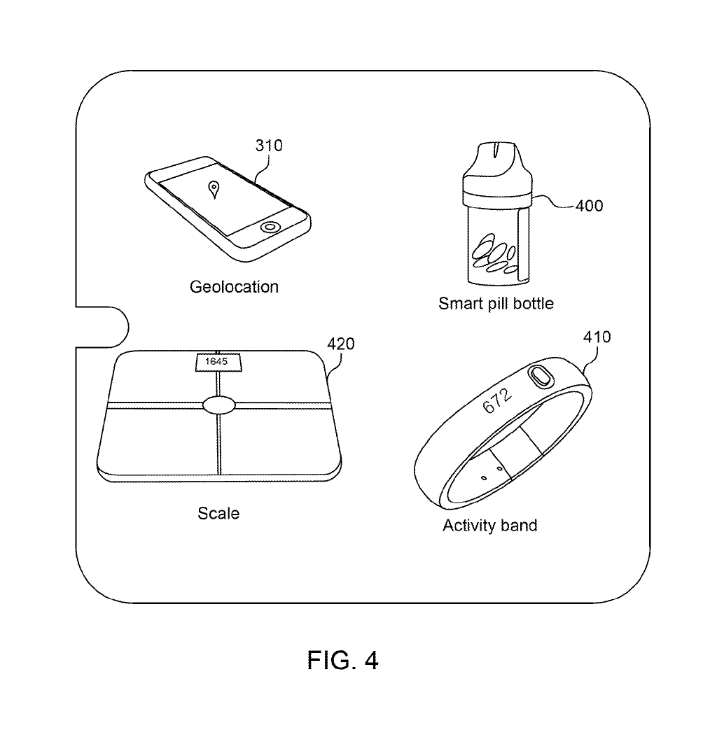

FIG. 4 illustrates example peripheral devices that may be used to collect relevant data and report this data to the system via mobile device 310 or otherwise. The smart medication bottle 400 (or smart pill bottle cap or other device for monitoring medication) may include sensors which detect when medication has been removed from or added to the bottle. The smart medication bottle 400 may be configured to communicate, e.g. through a BLUETOOTH® connection, to the system 200, information about medication adherence. For example, smart medication bottle 400 may communicate that the bottle has been accessed, a time at which the bottle was accessed, and/or an amount of medication removed from the bottle.

The activity band 410 may include sensors to monitor movement, sleeping patterns, vital signs, temperature and other activity associated with the wearer of the band. The activity band 410 may also include wireless or other communication capabilities and/or an access port such as a USB port to report information to the system 200.

Scale 420 may include sensors for measuring weight and/or body fat composition of the user, and may also include wireless or other communication capabilities and/or an access port such as a USB port to report weight and/or body fat composition information to the system 200.

Mobile device 310 may incorporate location tracking functionality or may receive such data from a GPS tracking device or other suitable geolocation peripheral (not shown). Mobile device 310 may also include wireless or other communication capabilities to report information to the system 200. In some implementations, geolocation and reporting of geolocation information to the system 200 may be performed by a standalone peripheral (not shown) which is separate from mobile device 310. Those having skill in the art will appreciate that other types of data capture devices may be used to record and report relevant data to system 200.

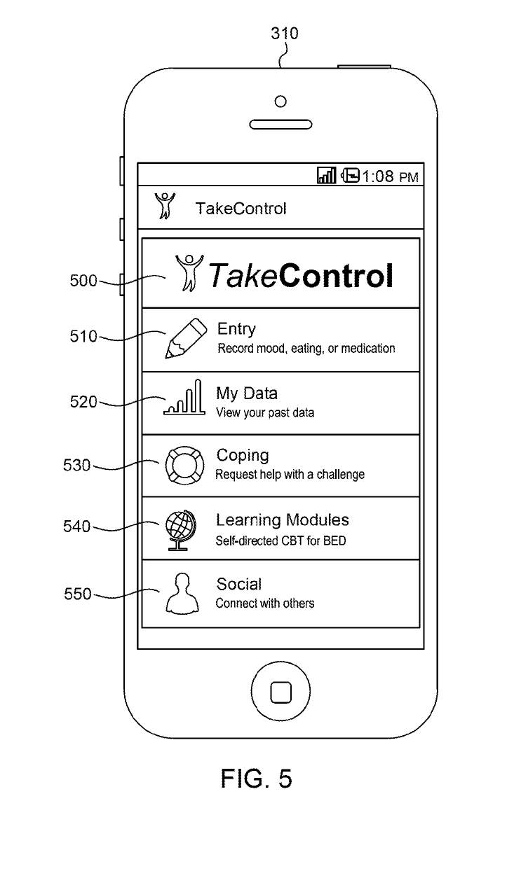

FIGS. 5-15B illustrate example screen shots of an example embodiment operating on a mobile user device.

FIG. 5 illustrates an example screen 500 presented to a user via mobile user device 310. The screen offers a menu of options for selection by the user. Options 510, 520, 530, 540, and 550 are provided for “Entry,” “My Data,” “Coping,” “Learning Modules,” and “Social,” Respectively. It is noted that in some implementations one or more of these options may be omitted and/or other options may be provided.

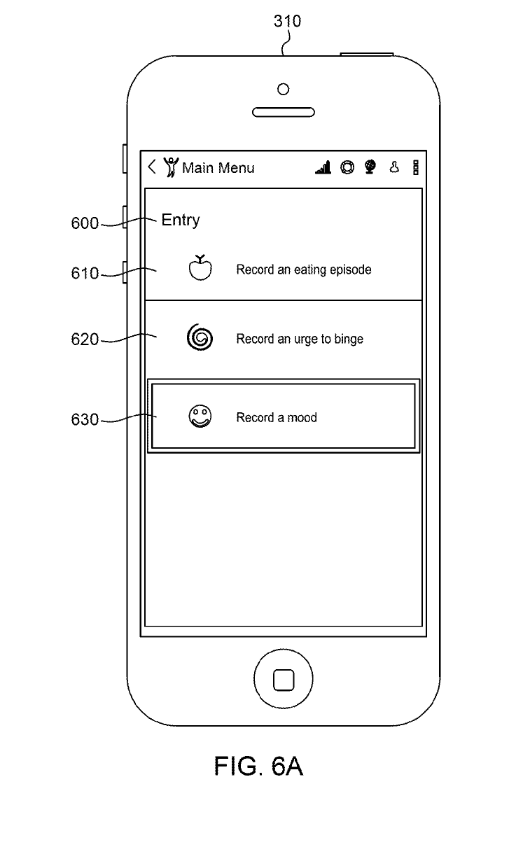

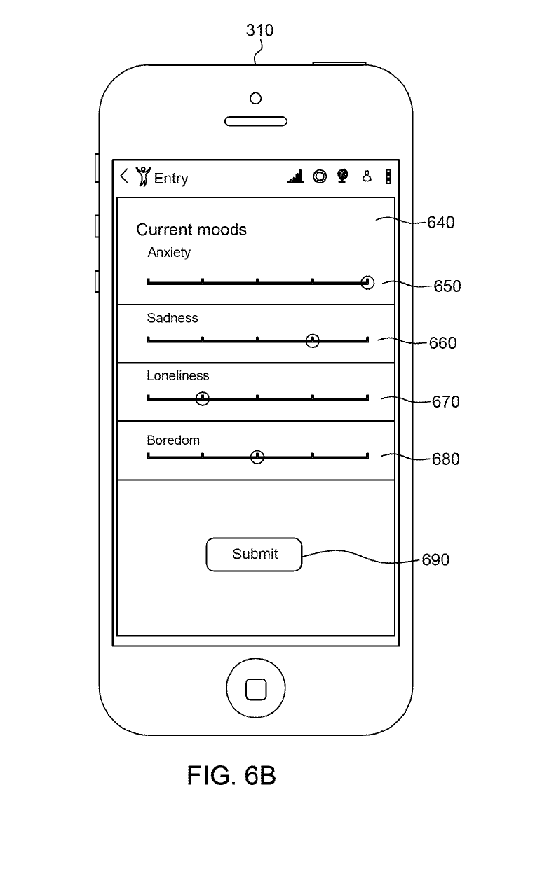

A user may select option 510, by touching the option on the touch screen of mobile device 310, although other input mechanisms may be used in various implementations. Selecting option 510 may take the user to a subsequent screen, such as screen 600 shown in FIG. 6A, that allows the user to enter relevant data.

FIG. 6A illustrates an example screen 600 which may follow user selection of option 510 “Entry.” Screen 600 offers a menu of options for recording data relevant to management of BED. Options 610, 620, and 630 are provided for “Record an eating episode,” “Record an urge to binge,” and “Record a mood” respectively. A user may select option 630 to record a current mood for example by selecting this option on the touch screen of mobile device 310. Selecting option 630 may take the user to a subsequent screen, such as screen 640 shown in FIG. 6B which allows the user to enter this data. The user may select option 610 to record eating a meal for example, which may take the user to a subsequent screen (not shown) which allows the user to enter this data. The user may likewise select option 620 to record an urge to binge, which may take the user to a subsequent screen (not shown) which allows the user to indicate an urge to binge at that time. It is noted that in some implementations one or more of these options may be omitted and/or other options may be provided.

FIG. 6B illustrates an example screen 640 following user selection of option 630 “Record a mood.” The user is presented with a list of moods and sliding scales for allowing the user to indicate the intensity of the mood. In this example, sliders 650, 660, 670, and 680 correspond to anxiety, sadness, loneliness, and boredom respectively. It is noted that other moods or combinations of moods relevant to managing BED, such as hunger, anxiety, sadness, loneliness, boredom, embarrassment, and anger may be presented, and that other selection mechanisms, such as radio buttons or text entry may be used instead of or in addition to sliders. The user may use sliders 650, 660, 670, and 680 to select a relative intensity of each respective mood. After the user has chosen desired levels for each emotion using the sliders, he or she may press button 690 to submit this data to system 200.

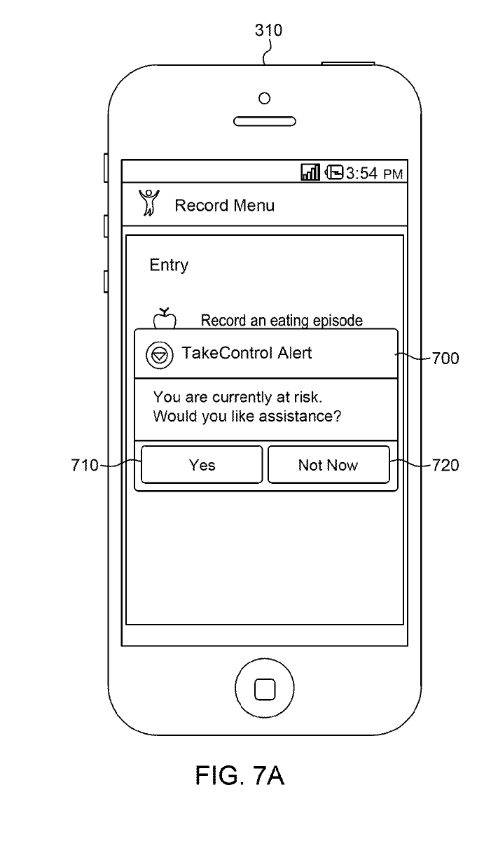

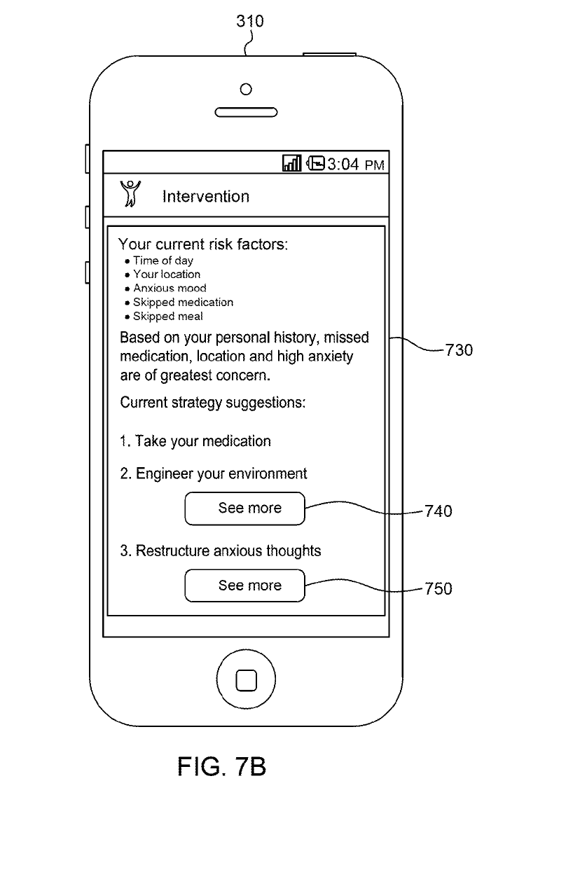

FIG. 7A illustrates an example of a preemptive alert 700. Preemptive alert 700 may be an EMI, and may be provided to the user when system 200 determines that the user is at risk of binging. System 200 may determine this risk based on an analysis of information received (via user input, peripheral devices, etc.) by system 200. For example, when the user enters a level of anxiety using slider 650 (FIG. 6B) exceeding a certain threshold, system 200 may determine that a risk of binging exists and transmit alert 700 to the user via mobile device 310. The user may accept or decline assistance using buttons 710 and 720 respectively.

The risk determination may depend upon a combination of factors. For example, the system may determine that a risk exists when the user indicates a threshold level of anxiety and the system determines that the user has failed to take prescribed medication as reported by peripheral device 320 (FIG. 3.) Peripheral device 320 may be a smart medication bottle 400 (FIG. 4) in this example. Those skilled in the art will appreciate that many other combinations of data input to system 200 either by the user, implicitly (e.g. time of day,) or from various peripheral devices may be used in determining a risk of binging.

The risk determination may be determined adaptively, via machine learning, expert systems, statistical analysis, or other suitable techniques and as further described herein. For example, if the user records an urge to binge using option 630 (FIG. 6A,) system 200 may correlate this urge with other collected input data such as anxiety level and medication adherence in order to preemptively provide alert 700 to the user in the future based on related inputs. Thus system 200 may learn the specific binge triggers for the user and provide customized treatment. The system may thus also iteratively refine the customized treatment over time as more data is gathered. This may have the advantage of automatically tailoring the treatment to each individual user, or of progressively modifying the parameters of the treatment as the user's BED progresses or improves over time.

FIG. 7B illustrates an example screen 730 which may follow user selection of button 710. Screen 730 may display a list of current risk factors for triggering BED and may identify one or more risk factors determined to be of greatest concern. Screen 730 may also present suggested strategies for coping with the identified risk factors, and may prompt the user to request additional information regarding risk factors and or coping strategies.

The listed risk factors, suggested strategies, and information provided to the user for coping with the current risk may depend upon the factors used in the determination of risk of binging as discussed above regarding FIG. 7A. Using the example above for instance, if the risk is identified as resulting from a combination of an elevated anxiety level and missed medication, the system may list these risk factors for the user, and may suggest taking medication or engaging in anxiety reducing behaviors as strategies for resisting the urge to binge.

In the example of FIG. 7B, screen 730 lists time of day, location, anxious mood, skipped medication, and skipped meal as current risk factors, and identifies missed medication, location, and high anxiety as of greatest concern based upon personal history and inputs to system 200. Screen 730 also lists strategies for coping with BED in view of the identified risk factors, including taking medication, engineering the environment, and restructuring anxious thoughts. The user is prompted to seek further information if desired regarding engineering the environment and restructuring anxious thoughts by selecting buttons 740 or 750 respectively. These risk factors and coping strategies may be identified by analyzing data provided to the system by the user and collected by sensors such as peripheral devices as further discussed herein.

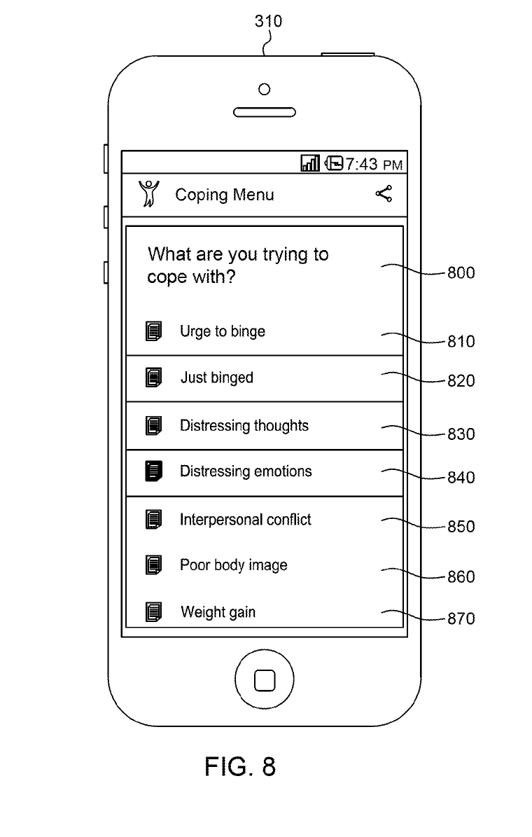

FIG. 8 illustrates an example screen 800 for requesting assistance with a BED challenge which may follow user selection of option 530 (“coping” FIG. 5).

Recognizing that users may encounter negative physical, emotional, social, and other experiences which may not be detected or anticipated by system 200, screen 800 allows the user to request help coping with various challenges on their own, without prompting by the system 200. Screen 800 offers a menu of various BED challenges for selection by the user. Options 810, 820, 830, 840, 850, 860, and 870 are provided for “Urge to binge,” “Just binged,” “Distressing thoughts,” “Distressing emotions,” “Interpersonal conflict,” “Poor body image,” and “Weight gain” respectively. Other options (not shown) may include “Trigger food” for indicating encountering a food known to trigger BED. It is noted that in some implementations one or more of these options may be omitted and/or other options may be provided. System 200 may respond with a request for more information, an EMI, and/or other advice for coping with the selected BED challenge.

User selection from among the options of screen 800 may be captured by system 200 and stored in a database for analysis via machine learning, expert systems, statistical analysis, or other suitable techniques and as further described herein. For example, if the user indicates weight gain using option 870, system 200 may correlate this urge with other collected input data such as actual weight as recorded by a peripheral device such as scale 420 (FIG. 4). This data may be used to evaluate and provide insight into the relationship between actual weight gain and the user's perception of weight gain for example. This may have the advantage of tailoring and refining the parameters of the treatment.

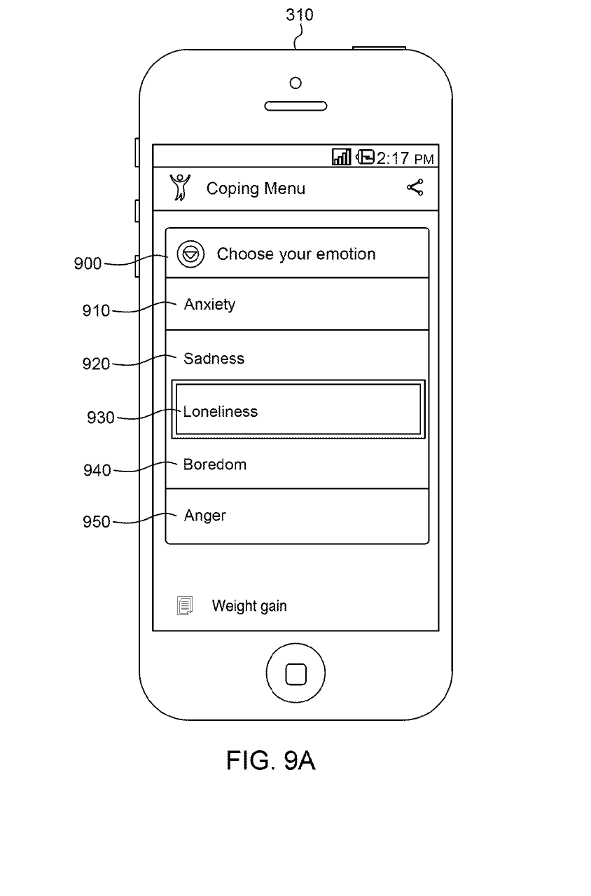

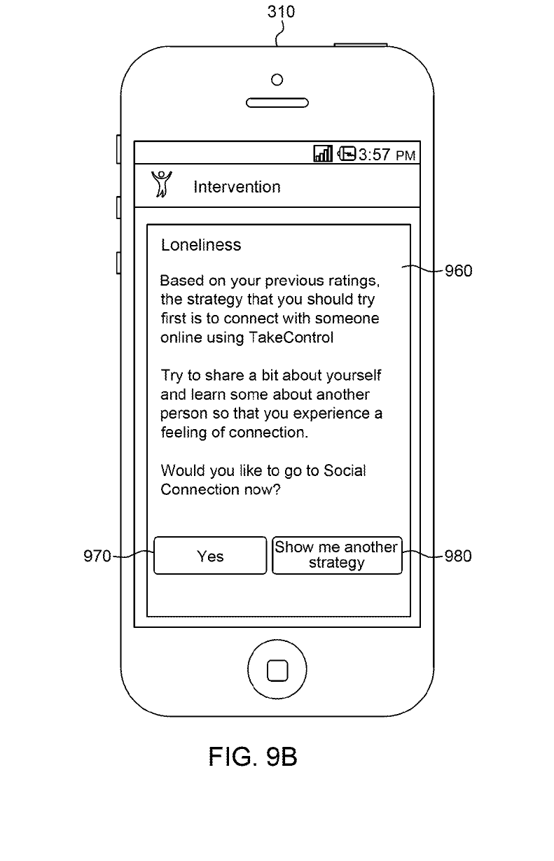

FIG. 9A illustrates an example screen 900 for providing assistance with a BED challenge which may follow user selection of option 840 (“Distressing emotions” FIG. 8). Screen 900 offers a menu of various distressing emotions for selection by a user using the touch screen. Options 910, 920, 930, 940, and 950 are provided for “Anxiety,” “Sadness,” “Loneliness,” “Boredom,” and “Anger” respectively. It is noted that in some implementations one or more of these options may be omitted and/or other options may be provided. By selecting an appropriate emotion that the user is experiencing, the user may obtain guidance for coping with this BED challenge.

FIG. 9B illustrates an example screen 960 following user selection of option 930 “Loneliness.” Screen 960 provides guidance to the user for coping with loneliness which may be tailored to the user's context, for example through analysis of data previously provided by the user or peripheral devices 315, 320, or 325 (FIG. 3) as further discussed herein. In this case, the user is prompted to engage in social interaction via an online forum. The user may accept this advice and be directed to appropriate online resources such as a forum by selecting button 970. The user may request another strategy for coping with this challenge by selecting button 980. Selection of buttons 930, 970, 980, or any other user interactions with system 200 may be logged by the system and analyzed to further tailor the treatment to the user, such as by correlating the user reported loneliness with the time of day for example.

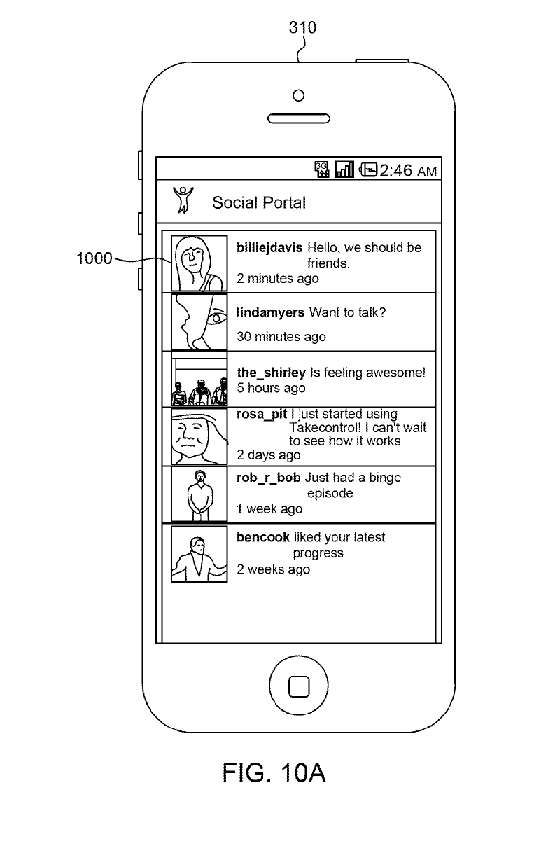

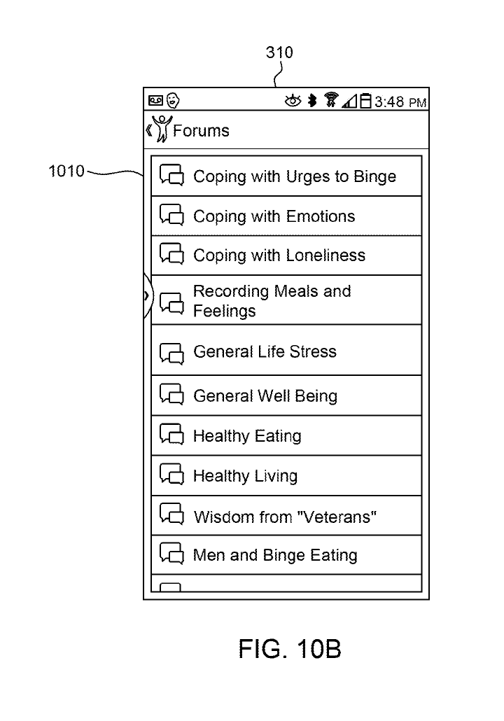

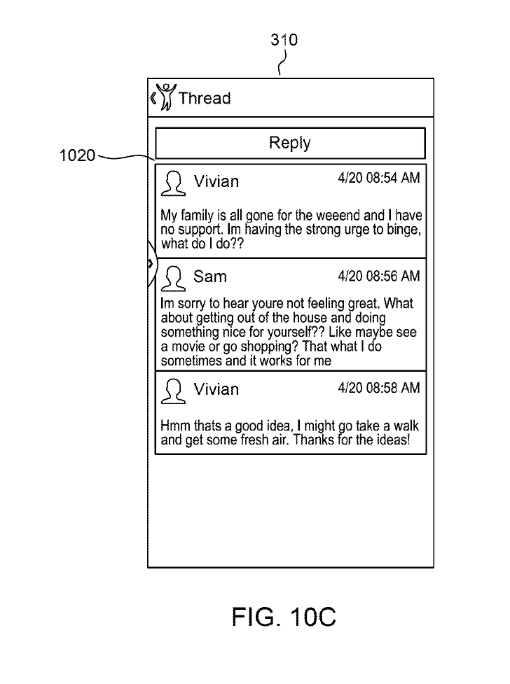

FIGS. 10A, 10B, and 10C illustrate example screens 1000, 1010, and 1020 respectively for interacting with a social network in order to communicate with other users in the network.

System 200 may include a social network for interaction among various users, including patients suffering from BED, clinicians, family members, or any other parties relevant to the treatment of BED. In some implementations, system 200 may provide an interface to an existing social network or other social network which may not be specific to system 200 or BED. Screens 1000, 1010, and/or 1020 may follow user selection of option 550 “Social” (FIG. 5) for self-directed access to the social network. Screens 1000, 1010, and/or 1020 may also follow prompting by system 200 when the system determines that the user is at risk of binging based on a risk factor, such as following selection of button 970 (FIG. 9) as prompted by system 200 for coping with loneliness.

FIG. 10A illustrates a screen 1000 showing messages from various users of the social network. The user may select a message to read further message content, to interact with the sender of the message such as via text or e-mail, or to engage in other social networking related activities. FIG. 10B illustrates a screen 1010 showing a list of forums relating to various BED related topics. The user may select from among the forums to engage in dialog with other users in a bulletin-board discussion or other suitable format. FIG. 10C illustrates a screen 1020 showing an example dialog between a user and another example user of the social network presented as a message thread. Such messages may be exchanged using simple message service (SMS) messages, e-mail, or any other suitable mechanism or format. By engaging with other users via text messaging, forum chats, and other types of interactions, the user can efficiently engage with other BED sufferers, treatment specialists, and well-wishers in order to receive support, alleviate anxiety, or otherwise advance their treatment goals.

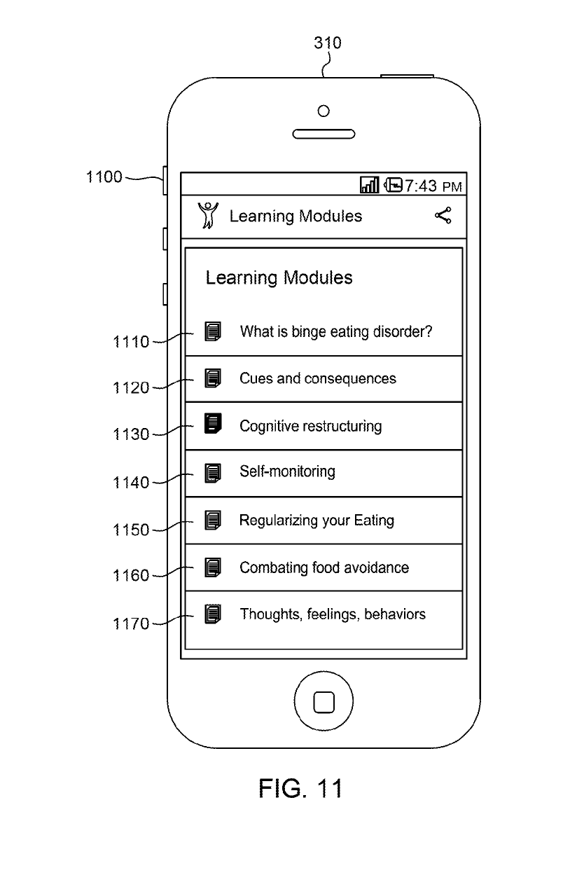

FIG. 11 illustrates an example screen 1100 for accessing learning modules for self-education regarding BED topics. Users may wish to access learning modules on their own for general learning or in response to encountering negative physical, emotional, social, and other experiences which may not be detected or anticipated by system 200. In this case, screen 1100 may follow user selection of option 540 (“Learning modules” in FIG. 5). Users may also be prompted to engage with a learning module by system 200 following detection of a risk of binging. In this case, screen 1100 may follow user selection of a prompt presented by system 200 such as screen 730 (FIG. 7B.) In either case, user's selections and other interactions with the system may be recorded by system 200 and used for further refinement of binging risk prediction and treatment of BED, such as by correlating the selections and interactions with other data which is input by the user, input from peripheral devices or determined implicitly.

In this example, options 1110, 1120, 1130, 1140, 1150, 1160, and 1170 correspond to learning modules relating to the topics “What is binge eating disorder,” “Cues and consequences,” “Cognitive restructuring,” “Self-monitoring,” “Regularizing your eating,” “Combatting food avoidance,” and “Thoughts, feelings, behaviors” respectively. Other options (not shown) may include “Initial interview”, “Orientation”, “Alternatives to binge eating”, “Strategies for changing binge eating cues”, “Identifying automatic thoughts”, and “Restructuring thoughts”. It is noted that in some implementations one or more of these options may be omitted and/or other options may be provided. Following selection of an option, other screens may be presented to the user with further information pertaining to the relevant topic.

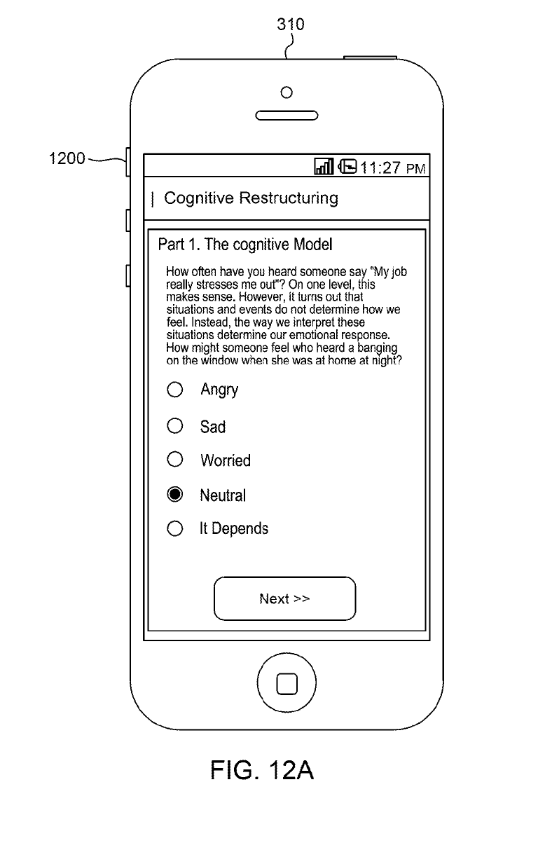

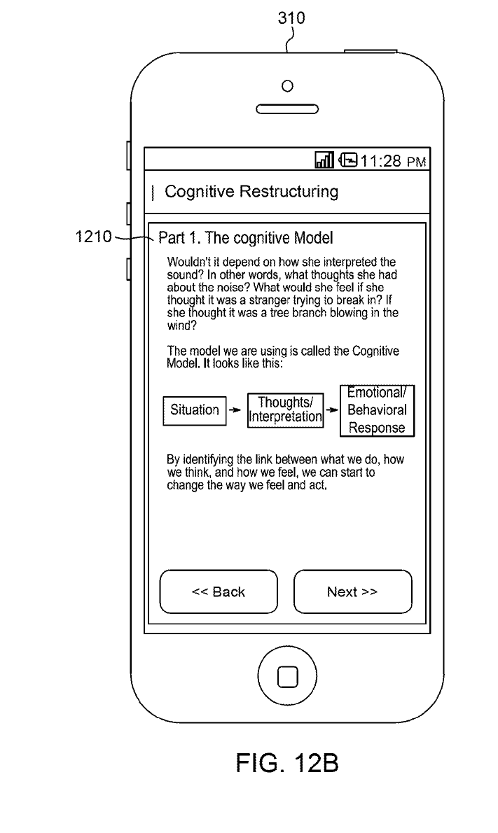

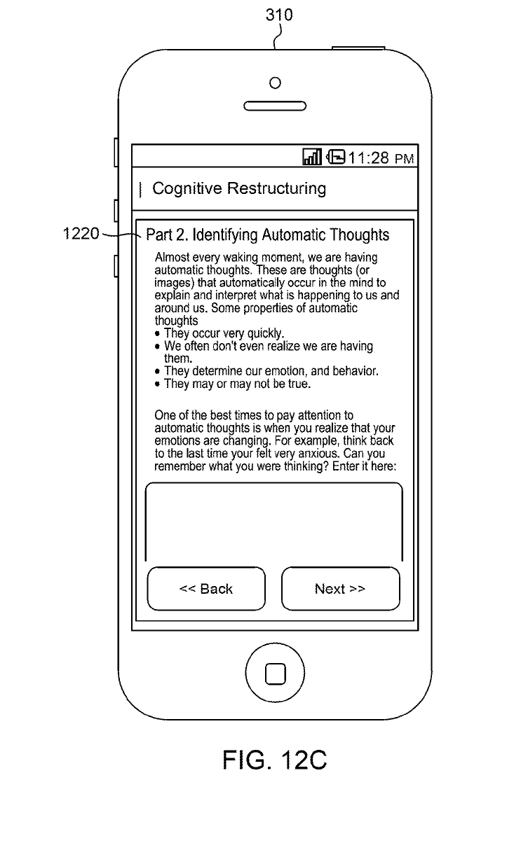

FIGS. 12A, 12B, and 12C illustrate an example series of screens 1200, 1210, and 1220 which may follow either user selection of option 1130 “Cognitive restructuring” or button 750 (“Restructure anxious thoughts” FIG. 7B). In this example, the user may be presented with information about cognitive restructuring, which may include background information and interactive learning. Related data may also be collected from the user for improving analysis of the user's context or for improving the function of the system. It is noted that different screens (not shown) having appropriate content may be presented to the user upon selection of other learning modules from screen 1100 (FIG. 11).

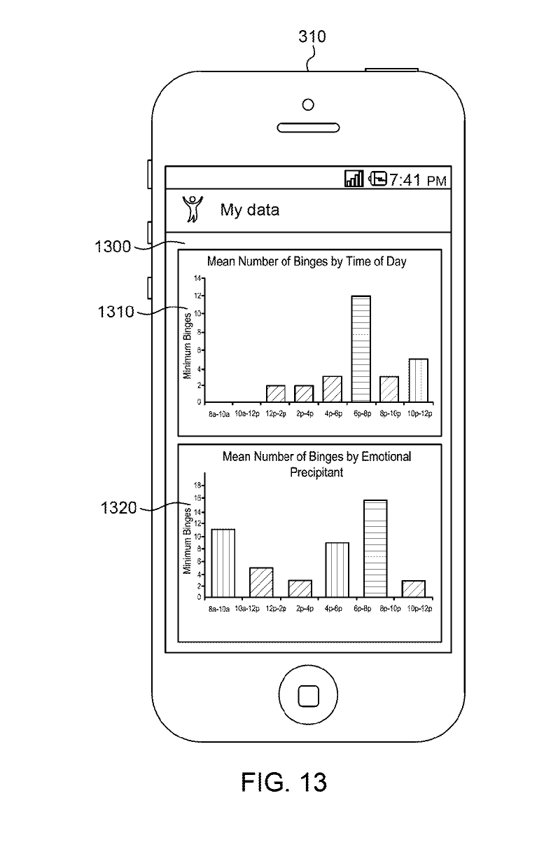

FIG. 13 illustrates an example screen 1300, which may follow user selection of option 520 (“My Data” FIG. 5).

System 200 may provide data visualization based on collection and/or analysis of the data input by the user, from various peripheral devices, or determined implicitly as discussed herein. Data visualization may assist the user or a clinician to gain insight into the user's various behaviors, habits, or BED triggers, the progress of the treatment, or the functioning of system 200 for example.

Screen 1300 displays bar charts showing an analysis of user data. Chart 1310 is an analysis of mean number of eating binges by time of day for the user, and chart 1320 is an analysis of mean number of binges by emotional precipitant. These charts may be generated by analyzing the various inputs to system 200 as discussed herein, and other types of user data may be presented as appropriate. Such charts may have the advantage of providing the user with insight into their behavior and triggers for more effective self-management of BED.

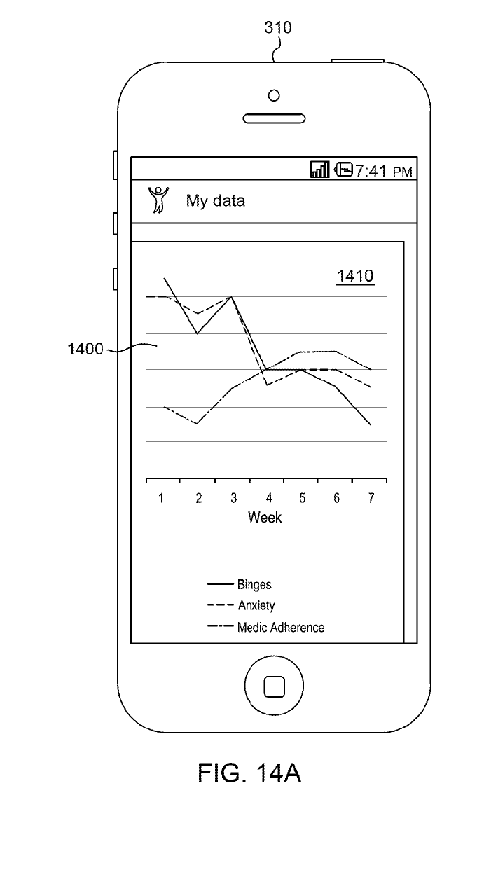

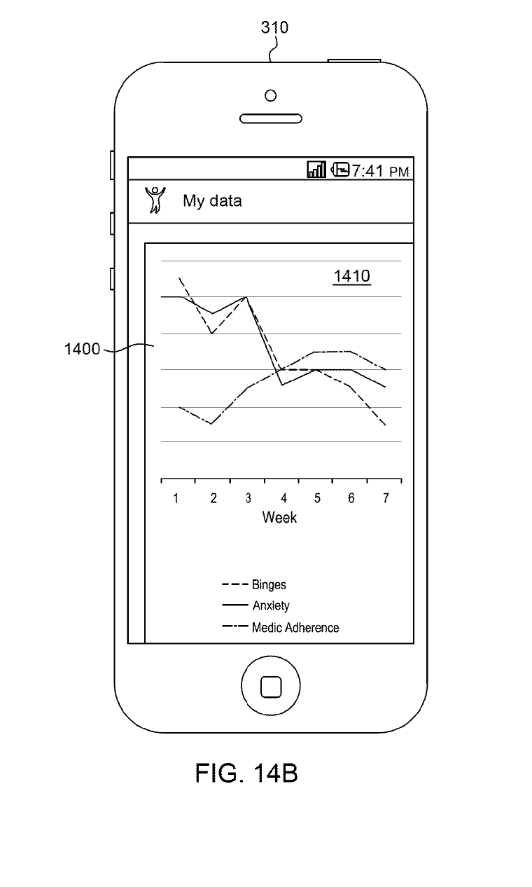

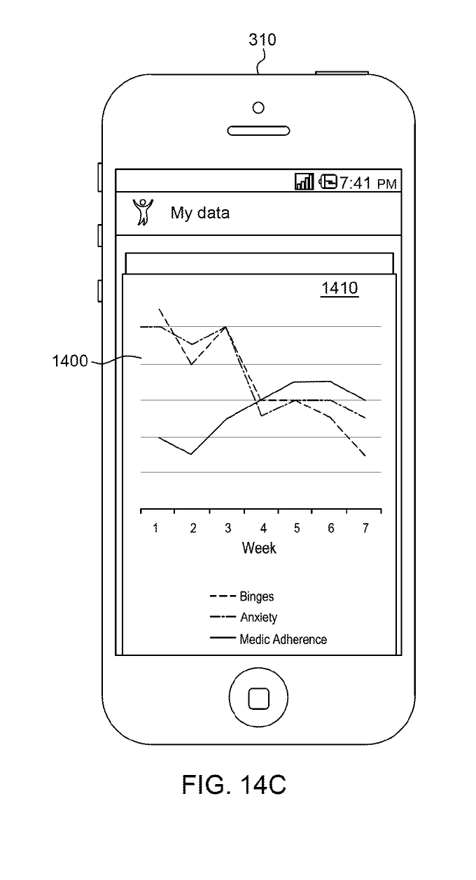

FIGS. 14A, 14B, and 14C illustrate another example screen 1400 which may follow user selection of option 520 “My Data” (FIG. 5). In some implementations the user may be able to switch between screens 1300 and 1400 by swiping a touchscreen of mobile device 310 or by another selection mechanism (not shown). Screen 1400 displays a line chart showing an analysis of user data. Line chart 1410 illustrates the number of binges per week, level of anxiety per week, and medicine adherence per week as reported by the user or captured by peripheral devices. Trend lines for binges, anxiety and medication adherence are highlighted respectively in FIGS. 14A, 14B, and 14C. Other types of user data may be presented as appropriate. Such charts may have the advantage of providing the user with insight into trends in their behavior over time and into the correlation of behaviors, emotions, medication adherence, and/or other factors for more effective management of BED.

It is noted that the data presentations in FIGS. 13 and 14 are exemplary, and other data, data analyses, or presentation formats may be used as desired.

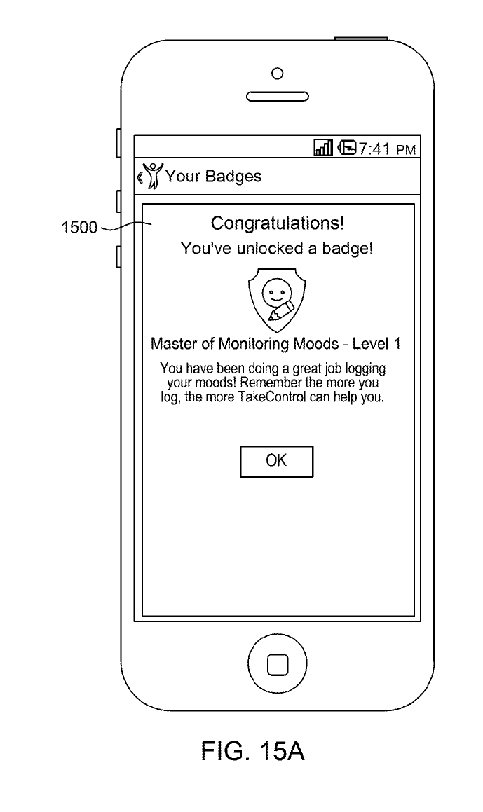

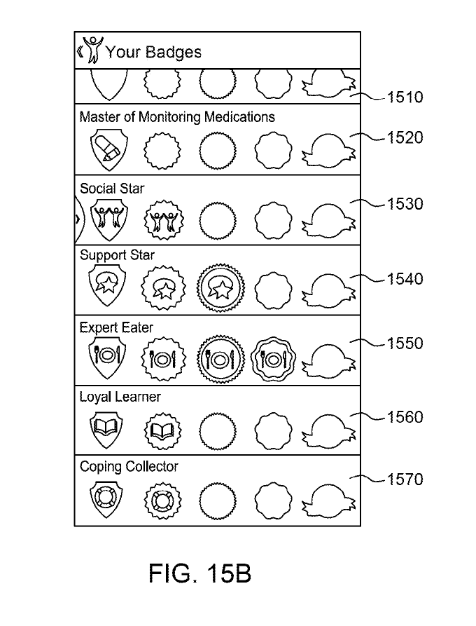

FIGS. 15A and 15B illustrate example screens 1500 and 1510, which relate to incentives provided to encourage certain user actions. In a typical treatment scenario, user persistence with a treatment regimen for BED may taper off after an initial period of compliance. Accordingly, it may be desirable to incorporate incentives and gamification into a course of treatment for BED to increase user engagement with system 200.

Incentives such as awards and recognition may be presented to a user following a particular desired user action or period of user compliance with desired actions such as logging data with system 200 using mobile device 310, engaging other users with social networking, adhering to a course of medication, and so forth. Screen 1500 informs the user that they have been awarded a “badge” by the system following the user's consistent logging of moods (using option 510 (FIG. 5) and screens 600 and 640 (FIGS. 6A and 6B) for example.) Screen 1500 provides recognition and encouragement to the user which may reinforce desired behavior. Other similar screens may be used for reinforcing other desired behaviors.

Screen 1510 displays accumulated “badges” awarded to the user by the system for engaging in desired behaviors. For example, rows 1520, 1530, 1540, 1550, 1560, and 1570 display badges awarded for engaging in desired behaviors relating to medication monitoring (using option 510 (FIG. 5) to record taking medication for example,) social engagement (using option 550 (FIG. 5) and screen 1000 (FIG. 10) to engage with the social network features of system 200 for example,) providing support to others (for example, using the social network functionality), diet management (using option 510 (FIG. 5) to record eating a meal for example,) engagement with learning modules (using option 540 (FIG. 5) and screens 1100, (FIGS. 11) 1200, 1210, and 1220 (FIGS. 12A, 12B, and 12C, respectively) to learn more about cognitive restructuring for example,) and engagement with the coping functionality (using option 530 (FIG. 5) and screens 800 (FIGS. 8) 900 and 960 (FIG. 9) to request help coping with loneliness for example) respectively.

It is noted that incentives may be awarded for any other desired behaviors and that incentives may take other suitable forms, such as points, level attainment, leaderboards among other users, monetary incentives, and the like. Gamification and reinforcement using incentives in this way may increase user engagement and persistence with the system and improve treatment outcomes.

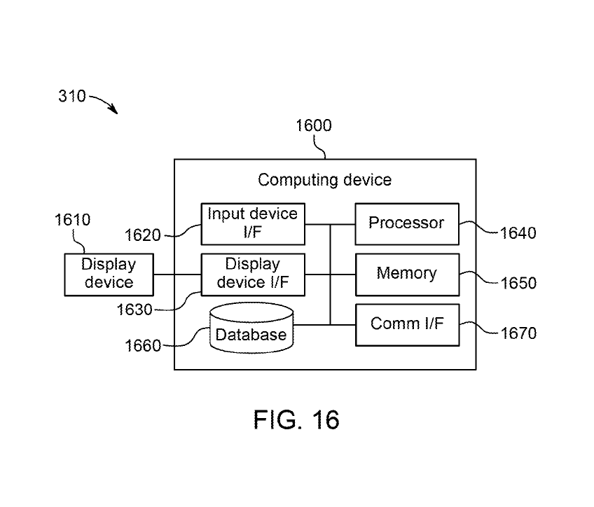

FIG. 16 is a diagram illustrating an example architecture for mobile user device 310. Device 310 may include a computing device 1600 and a display device 1610, which may be integrated into one device as desired. Computing device 1600 may include an input interface 1620, display device interface 1630, processor 1640, memory 1650, database 1660, and communication interface 1670. Device 310 may be a smartphone, tablet computer, notebook or laptop computer, personal computer, media player, or any other suitable computing device. Database 1660 may include a non-transitory computer readable medium containing instructions which when executed by processor 1640 cause the device to carry out functions of system 300 as further discussed herein.

Disclosed herein are processor-executable methods, computing systems, devices, and related technologies for managing binge eating disorders (BEDs). However it is noted that these may be applicable to other disorders, including drug addiction.

Various implementations are described herein using certain commercially available devices by way of example only, and it is noted that other implementations are possible, mutatis mutandis, using any appropriate architecture, equipment, and/or computing or communications environment.

As used herein, the term “processor” broadly refers to and is not limited to a single- or multi-core processor, a special purpose processor, a conventional processor, a Graphics Processing Unit (GPU), a digital signal processor (DSP), a plurality of microprocessors, one or more microprocessors in association with a DSP core, a controller, a microcontroller, one or more Application Specific Integrated Circuits (ASICs), one or more Field Programmable Gate Array (FPGA) circuits, any other type of integrated circuit (IC), a system-on-a-chip (SOC), and/or a state machine.

As used to herein, the term “computer-readable medium” broadly refers to and is not limited to a register, a cache memory, a ROM, a semiconductor memory device (such as a D-RAM, S-RAM, or other RAM), a magnetic medium such as a flash memory, a hard disk, a magneto-optical medium, an optical medium such as a CD-ROM, a DVDs, or BD, or other type of device for electronic data storage.

Although the methods and features described above with reference to FIGS. 4-16 are described above as performed using the example architecture of FIG. 3, the methods and features described above may be performed, mutatis mutandis, using any appropriate architecture and/or computing environment. Although features and elements are described above in particular combinations, each feature or element can be used alone or in any combination with or without the other features and elements. For example, each feature or element as described above with reference to FIGS. 3-16 may be used alone without the other features and elements or in various combinations with or without other features and elements. Sub-elements of the methods and features described above with reference to FIGS. 3-16 may be performed in any arbitrary order (including concurrently), in any combination or sub-combination.

What is claimed is:

1. A system for managing a binge eating disorder, the system comprising:
a first mobile device, comprising a processor, a memory, and a display, configured to receive input from a user regarding eating habits and psychological information;
at least one peripheral device, operatively coupled to the first mobile device, configured to record data and transmit information to the first mobile device;
the first mobile device further configured to transmit user information and the recorded data from the peripheral device, to a server; and
the first mobile device configured to generate and display, to the user, notifications regarding the users eating habits.

2. The system of claim 1, wherein the notifications are generated based on the input or the recorded data.

3. The system of claim 1, further comprising a clinical portal device configured to access the information stored in the server.

4. The system of claim 1, further comprising a second mobile device, configured to communicate with the first mobile device via a social networking application.

5. The system of claim 1, wherein the at least one peripheral device comprises an activity band, a web-connected scale, a smart pill bottle, a smart pill bottle cap, or a geolocation device.

6. The system of claim 1, wherein the notifications are generated based on an analysis of the input or the recorded data and other input or recorded data received from other users.

7. A mobile device for managing a binge eating disorder, comprising:
a processor,
a memory,
a display, and
an input interface;
the input interface configured to receive user behavioral data and user context data;
the user behavioral data including information regarding a mood, a physical state, an urge to binge, a binging episode, a meal, a social interaction, an interpersonal conflict, or a desire for guidance;
the user context data including information about a user location, activity, body weight, body composition, or medication compliance; and
wherein the device is configured to display an alert relating to the binge eating disorder based on the user behavioral data, the user context data, or both the user behavioral data and the user context data.

8. The mobile device of claim 7, wherein the alert comprises an intervention related to the behavioral data or the context data.

9. The mobile device of claim 7, wherein the input interface is configured to receive manual input of the behavioral data or the context data.

10. The mobile device of claim 7, wherein the input interface is configured to receive the context data from a sensor.

11. The mobile device of claim 10, wherein the sensor comprises an activity monitor, a scale, a body composition monitor, a medication monitoring device, a smart pill bottle, a smart pill bottle cap, or a geolocation device.

12. The mobile device of claim 10, wherein the input interface is configured to receive the context data automatically.

13. The mobile device of claim 7, further configured to display the alert to the user following an occurrence of a trigger.

14. The mobile device of claim 13, further configured to identify the occurrence of the trigger by correlating current user behavior and current user context.

15. The mobile device of claim 13, further configured to identify the trigger by correlating prior user behavior and prior user context.

16. The mobile device of claim 13, wherein the trigger comprises a combination of one or more behavioral data or context data.

17. The mobile device of claim 13, further configured to transmit a message to a second user via a social network in response to the trigger.

18. The mobile device of claim 7, further configured to transmit the user behavioral data and user context data over a communications network to a server for analysis.

19. The mobile device of claim 7, further configured to analyze the user behavioral data and/or user context data using the processor to generate the alert.

20. The mobile device of claim 18, wherein the device receives the alert for display from the server.
